(19) United States
(12) Patent Application Publication
(10) Pub. No.: US 2026/0331078 A1
(43) Pub. Date: Oct. 22, 2026

(54) Digital Display Apparatus With Adjustable Dual Refresh Frequencies

(71) Applicant: Aleddra Inc., Renton, WA (US)

(72) Inventors: Chia-Yiu Maa, Bellevue, WA (US); Li-Jyuan Luo, Taipei City (TW)

(73) Assignee: Aleddra Inc., Renton, WA (US)

(21) Appl. No.: 19/726,737

(22) Filed: Jun. 30, 2026

Related U.S. Application Data
(63) Continuation-in-part of application No. 19/253,662, filed on Jun. 27, 2025, now Pat. No. 12,731,525.
Application No. 19/253,662 is a continuation-in-part of application No. 19/065,863, filed on Feb. 27, 2025, now Pat. No. 12,725,559.
Application No. 19/065,863 is a continuation-in-part of application No. 18/791,703, filed on Aug. 1, 2024, now Pat. No. 12,374,263.
(63) Continuation-in-part of application No. 19/227,439, filed on Jun. 3, 2025.

(51) Int. Cl. G09G 5/36

(52) U.S. Cl. CPC G09G 5/363; G09G 2310/08

(57) ABSTRACT

A digital display apparatus includes a controller and a digital pixel panel. A plurality of pixels of the digital pixel panel are divided into first and second groups of pixels. The controller displays a graphic content via the digital pixel panel. The controller operates the first group of pixels at a first flicker-free refresh frequency (F1) ≥ 60Hz and the second group of pixels at a second flicker-free refresh frequency (F2) > F1. When the graphic content is viewed by a subject, synchronized gamma oscillations at a differential frequency equal to |2F1 – F2| (between 20Hz and 45Hz) are induced endogenously in at least one brain region of a subject. The controller supports at least two differential frequencies and switching from one differential frequency to another among the at least two differential frequencies to strengthen synchronized gamma oscillations in the at least one brain region.

### BACKGROUND

### CROSS-REFERENCE TO RELATED APPLICATIONS

The present disclosure is a continuation-in-part (CIP) of US Patent Application 19/253,662, filed 27 Jun. 2025, which itself is a CIP of US Patent Application 19/065,863, filed 27 Feb. 2025, which itself is a CIP of US Patent Application 18/791,703, filed 1 Aug. 2024. The present disclosure is also a CIP of US Patent Application 19/227,439, filed 3 Jun. 2025. Contents of aforementioned applications are herein incorporated by reference in their entirety.

### TECHNICAL FIELD

The present disclosure pertains to the field of digital display apparatus and, more specifically, proposes digital display apparatus with adjustable dual refresh frequencies.

### DESCRIPTION OF RELATED ART

In US Patent Application 19/253,662, filed 27 June 2025, it introduced a digital display apparatus comprising a controller and a digital pixel panel. The pixels of the digital pixel panel are divided into a first group of pixels and a second group of pixels. The controller is configured to operate the first group of pixels at a first flicker-free refresh frequency F1 and to operate the second group of pixels at a second flicker-free refresh frequency F2 > F1. When the graphic content displayed on the digital pixel panel is viewed by a subject, an invisible visual simulation at a harmonic frequency equal to |2F1 – F2| is induced endogenously in a brain of the subject. By properly choosing F1 and F2, and subsequently HF, the digital display apparatus offers the benefits of preventing or improving Alzheimer's disease or memory recall. One distinct feature of this digital display apparatus is that it has a fixed harmonic frequency.

Recent studies have shown that the optimal stimulation frequency for entraining gamma waves in human brain varies from person to person and may change as a person aged. For example, one study shows older adults have stronger gamma entrainment at 32Hz or 34Hz. Another study shows young adults have stronger gamma entrainment at 34-38Hz. These results raise the question of the effectiveness of using only 40Hz gamma stimulation for treating AD for people of all ages.

The present disclosure enhances the invention introduced in US Patent Application 19/253,662, filed 27 June 2025 by supporting multiple harmonic frequencies and enabling a user to choose among multiple harmonic frequencies according to his/her optimal gamma stimulation frequency.

### SUMMARY

In one aspect, the digital display apparatus comprises a controller and a digital pixel panel. A plurality of pixels of the digital pixel panel are divided into a first group of pixels and a second group of pixels. The controller is configured to display a graphic content via the digital pixel panel. The controller is configured to operate the first group of pixels at a first flicker-free refresh frequency (F1) ≥ 60Hz. The controller is configured to operate the second group of pixels at a second flicker-free refresh frequency (F2) > F1. When the graphic content displayed on the digital pixel panel is viewed by a subject, synchronized gamma oscillations at a differential (harmonic) frequency equal to |2F1 – F2| are induced endogenously in at least one brain region of a subject. Differential frequency is invisible because it is induced endogenously in the brain of the subject. The digital display apparatus does not emit such differential frequency directly. |2F1 – F2| refers to the absolute value of the difference of 2F1 and F2. In other words, their difference at 40Hz and -40Hz are the same as far as inducing an endogenous stimulation in the brain of the subject is concerned.

The differential frequency of this digital display apparatus is between 20Hz and 45Hz. In practice, differential frequency may be more narrowly chosen to be between 30Hz and 40Hz, thus generating gamma stimulation in the brain of the subject for treating or preventing Alzheimer's disease.

Moreover, the controller is configured to support at least two differential frequencies. The controller is configured to support switching from one differential frequency to another among the at least two differential frequencies to strengthen synchronized gamma oscillations in the at least one brain region.

It is to be noted that switching from one differential frequency to another doesn’t depend on the content being displayed on the digital pixel panel. Rather, the switching would depend on the optimal gamma stimulation responsiveness of the subject and most likely be chosen manually by the subject. Therefore, in some embodiments, switching from one differential frequency to another among the at least two differential frequencies is triggered manually.

In some embodiments, one of the at least two differential frequencies is 40Hz. There are multiple combinations of F1 and F2 for achieving 40Hz differential frequency, e.g., F1=80Hz and F2=120Hz, or F1=80Hz and F2=200Hz. Another of the at least two differential frequencies may be 38Hz, 36Hz, 34Hz, or 32Hz.

In some embodiments, one of the at least two differential frequencies is 32Hz. There are multiple combinations of F1 and F2 for achieving 32Hz differential frequency, e.g., F1=80Hz and F2=128Hz, or F1=80Hz and F2=192Hz. Another of the at least two differential frequencies may be 40Hz, 38Hz, 36Hz, and 34Hz.

In some embodiments, from the perspective of a subject (e.g., a user) viewing the digital pixel panel, the first group of pixels occupies the top section of the digital pixel panel and the second group of pixels occupies the bottom section of the digital pixel panel as shown in FIG. 2. The white dots in FIG. 2 denote the pixels in the first group of pixels and the black dots denote the pixels in the second group of pixels. Other layout arrangements for the first group of pixels and the second group pixels are possible. However, separating the first group of pixels and the second group of pixels in disjoint regions simplifies the hardware design of the controller and minimizes the perceptibility of refresh frequency discrepancy between the first group of pixels and the second group pixels.

In some embodiments, the controller having two display channels. The first display channel feeds graphic content to the first group of pixels and the second display channel feeds graphic content to the second group of pixels.

In some embodiments, the digital pixel panel comprises a first physical digital pixel panel and a second physical display panel. In which case, the controller is configured to operate the first physical digital pixel panel at F1 refresh frequency and the second physical display pixel panel at F2 refresh frequency. Examples include but are not limited to dual-monitor computer systems, dual-screen smartphones, and dual-lens digital goggles. With a dual-monitor computer system of the present disclosure, one monitor operates at F1 refresh frequency and the other monitor at F2 refresh frequency. The controller in this case may include the graphic controller of the host computer, the hardware responsible for refreshing the first monitors at F1, and the hardware responsible for refreshing the second monitor at F2. The controller determines which content will be displayed in the first monitor and which content will be displayed at the other monitor. Similarly, for a dual-screen smartphone of the present disclosure, one screen operates at F1 and the other screen at F2. For a dual-lens digital google of the present disclosure, one digital lens operates at F1 and the other digital lens at F2.

### BRIEF DESCRIPTION OF THE DRAWINGS

The accompanying drawings are included to aid further understanding of the present disclosure and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts an embodiment of the present disclosure.

FIG. 2 schematically depicts an embodiment of the present disclosure having the first group of pixels on the top section of the digital pixel panel and the second group of pixels on the bottom section of the digital pixel panel.

FIG. 3 schematically depicts an embodiment of the present disclosure as a computer system with dual monitors.

### DETAILED DESCRIPTION OF PREFERRED EMBODIMENTS

### OVERVIEW

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of digital display apparatuses having different form factors.

A digital display apparatus comprises a controller and a digital pixel panel. The pixels of the digital pixel panel are divided into a first group of pixels and a second group of pixels. The controller is configured to operate the first group of pixels at a first flicker-free refresh frequency F1 and to operate the second group of pixels at a second flicker-free refresh frequency F2 > F1. When the graphic content displayed on the digital pixel panel is viewed by a subject, synchronized gamma oscillations at a differential frequency equal to |2F1 – F2| are induced endogenously in at least one brain region of a subject. The differential frequency is between 20Hz and 45Hz. Moreover, the controller is configured to support at least two differential frequencies. The controller is configured to support switching from one differential frequency to another among the at least two differential frequencies to strengthen synchronized gamma oscillations in the at least one brain region.

### EXAMPLE IMPLEMENTATIONS

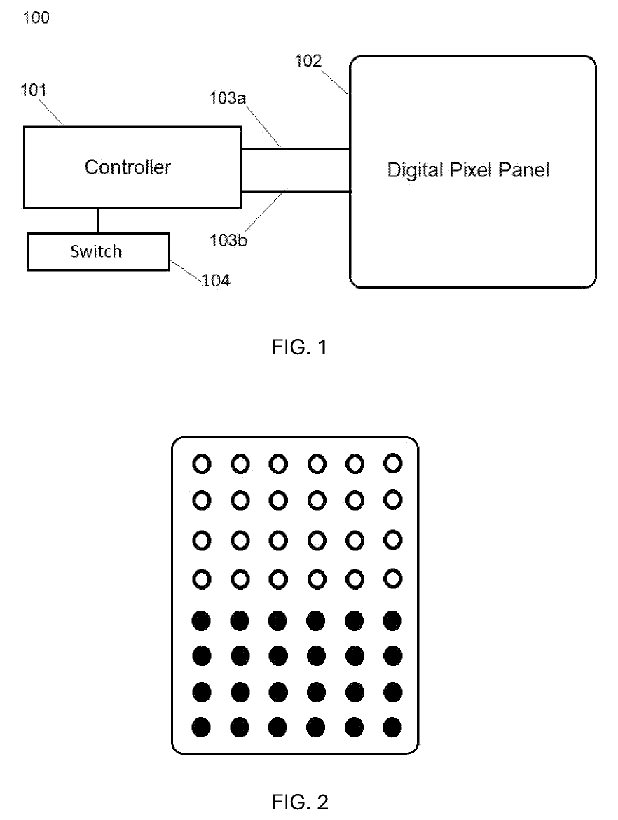

FIG. 1 shows an embodiment of the digital display apparatus of the present disclosure 100. It comprises a controller 101 and a digital pixel panel 102. The pixels of the digital pixel panel 102 are divided into a first group of pixels and a second group of pixels as shown in FIG. 2. The white dots in FIG. 2 denote the pixels in the first group of pixels and the black dots denote the pixels in the second group of pixels. Controller 101 has two channels 103a, 103b. Controller 101 feeds graphic content through channel 103a to the first group of pixels which are operating at 80Hz refresh frequency. Similarly, controller 101 feeds graphic content through the second display channel 103b to the second group of pixels. Switch 104 can manually set the differential frequency to either 40Hz or 32Hz. When switch 104 is set to 40Hz, controller 101 operates the second group of pixels at 120Hz refresh frequency, thus |2x80Hz – 120Hz| = 40Hz. When switch 104 is set to 32Hz, controller 101 operates the second group of pixels at 128Hz refresh frequency, where |2x80Hz – 128Hz| = 32Hz.

When a subject views the digital pixel panel 102, the subject is exposed to both F1 (80Hz) and F2 (120Hz or 128Hz) refresh frequencies, thus synchronized gamma oscillations at 40Hz or 32Hz are induced endogenously in at least one brain region of the subject. The embodiment 100 is thus suitable for treating or preventing Alzheimer's disease. Other combinations may be chosen for F1 and F2 for achieving 40Hz and 32Hz differential frequencies. In some embodiments, switch 104 may support more than two differential frequencies. For example, switch 104 may support five differential frequencies: 40Hz, 38Hz, 36Hz, 34Hz, and 32Hz.

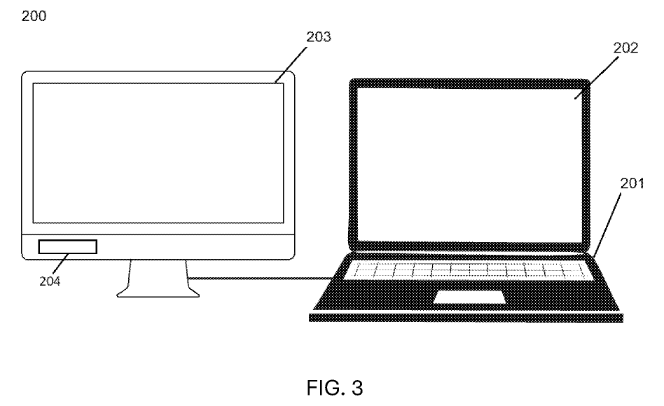

FIG. 3 shows an embodiment of the present disclosure 200 as a computer system with dual monitors. The laptop computer 201 has an integrated monitor 202 operating at 80Hz refresh frequency. The extended monitor 203 operates at either 120Hz or 128Hz refresh frequency, depending on the setting of switch 204. Switch 204 supports 40Hz and 32Hz differential frequencies. The (host) laptop computer controls and manages contents of both its integrated monitor 202 (the first physical digital pixel panel) and the extended monitor 203 (the second physical pixel panel). The content displayed in the integrated monitor 202 is not required to be correlated to the content displayed in the extended monitor 203. For example, the integrated monitor may show a Word document whereas the extended monitor may show a video. When a subject views the integrated monitor 202 and the extended monitor 203 simultaneously, the subject is exposed to both F1 (80Hz) and F2 (120Hz or 128Hz, depending on switch 204 setting) refresh frequencies, thus synchronized gamma oscillations are induced endogenously in at least one brain region of the subject. The embodiment 200 is thus suitable for treating or preventing Alzheimer's disease. Other combinations may be chosen for F1 and F2.

FIG. 3 shows switch 204 is positioned on the extended monitor as a physical switch. Alternatively, switching (or configuration) among the at least two differential frequencies of embodiment 200 may be implemented via a display panel setting app on the (host) laptop computer 201 such that a user can use this display panel setting app to configure the differential frequency between F1 of the integrated monitor 202 and F2 of the extended monitor 203, i.e., without using a physical switch. Nonetheless, the configuration of the differential frequency via this display panel setting app is performed manually by the subject or a user of such embodiment. This display panel setting app may support two differential frequencies such as 40Hz and 32Hz, or more, e.g., five differential frequencies: 40Hz, 38Hz, 36Hz, 34Hz, and 32Hz.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A digital display apparatus comprising:
a controller; and
a digital pixel panel;
wherein:
a plurality of pixels of the digital pixel panel are divided into a first group of pixels and a second group of pixels,
the controller is configured to display a graphic content via the digital pixel panel,
the controller is configured to operate the first group of pixels at a first flicker-free refresh frequency (F1) ≥ 60Hz,
the controller is configured to operate the second group of pixels at a second flicker-free refresh frequency (F2) > F1,
when the graphic content displayed on the digital pixel panel is viewed by a subject, synchronized gamma oscillations at a differential frequency equal to |2F1 – F2| are induced endogenously in at least one brain region of the subject, with the differential frequency being between 20Hz and 45Hz,
the controller is configured to support at least two differential frequencies, and
the controller is configured to support switching from one differential frequency to another among the at least two differential frequencies to strengthen the synchronized gamma oscillations in the at least one brain region.

2. An apparatus of claim 1, wherein the switching from the one differential frequency to the another among the at least two differential frequencies is triggered manually.

3. An apparatus of claim 1, wherein one of the at least two differential frequencies is 40Hz.

4. An apparatus of claim 1, wherein one of the at least two differential frequencies is 32Hz.

5. An apparatus of claim 1, wherein the first group of pixels occupies a top section of the digital pixel panel and the second group of pixels occupies a bottom section of the digital pixel panel.

6. An apparatus of claim 1, wherein the controller is configured with two display channels comprising a first display channel and a second display channel, wherein the first display channel feeds the graphic content to the first group of pixels, and wherein the second display channel feeds the graphic content to the second group of pixels.

7. An apparatus of claim 1, wherein the digital pixel panel comprises a first physical digital pixel panel and a second physical display panel, and wherein the controller is configured to operate the first physical digital pixel panel at the F1 refresh frequency and the second physical display pixel panel at the F2 refresh frequency.

8. A method of operating a digital display apparatus comprising:
displaying, by a controller of the digital display apparatus, a graphic content via a digital pixel panel of the digital display apparatus, the digital pixel panel configured with a plurality of pixels divided into a first group of pixels and a second group of pixels; and
operating, by the controller:
the first group of pixels at a first flicker-free refresh frequency (F1) ≥ 60Hz, and
the second group of pixels at a second flicker-free refresh frequency (F2) > F1,
wherein, when the graphic content displayed on the digital pixel panel is viewed by a subject, synchronized gamma oscillations at a differential frequency equal to |2F1 – F2| are induced endogenously in at least one brain region of the subject, with the differential frequency being between 20Hz and 45Hz,
wherein the controller is configured to support at least two differential frequencies, and
wherein the controller is configured to support switching from one differential frequency to another among the at least two differential frequencies to strengthen the synchronized gamma oscillations in the at least one brain region.

9. A method of claim 8, wherein the switching from the one differential frequency to the another among the at least two differential frequencies is triggered manually.

10. A method of claim 8, wherein one of the at least two differential frequencies is 40Hz.

11. A method of claim 8, wherein one of the at least two differential frequencies is 32Hz.

12. A method of claim 8, wherein the first group of pixels occupies a top section of the digital pixel panel and the second group of pixels occupies a bottom section of the digital pixel panel.

13. A method of claim 8, wherein the controller is configured with two display channels comprising a first display channel and a second display channel, wherein the first display channel feeds the graphic content to the first group of pixels, and wherein the second display channel feeds the graphic content to the second group of pixels.

14. A method of claim 8, wherein the digital pixel panel comprises a first physical digital pixel panel and a second physical display panel, and wherein the controller is configured to operate the first physical digital pixel panel at the F1 refresh frequency and the second physical display pixel panel at the F2 refresh frequency.
